(19) United States
(12) Patent Application Publication
(10) Pub. No.: US 2013/0172571 A1
(43) Pub. Date: Jul. 4, 2013

(54) PROCESS TO PREPARE ETHYL 4-METHYL-2-(4-(2-METHYLPROPYLOXY)-3-CYANOPHENYL)-5-THIAZOLECARBOXYLATE

(71) Applicants: Amala Kompella, Hyderabad (IN); Venue Gopala Krishna Gampa, Hyderabad (IN); Kali Satya Bhujanga Rao Adibhatla, Hyderabad (IN); Venkaiah Chowdary Nannapaneni, Hyderabad (IN)

(72) Inventors: Amala Kompella, Hyderabad (IN); Venue Gopala Krishna Gampa, Hyderabad (IN); Kali Satya Bhujanga Rao Adibhatla, Hyderabad (IN); Venkaiah Chowdary Nannapaneni, Hyderabad (IN)

(73) Assignee: NATCO PHARMA LIMITED, Hyderabad (IN)

(21) Appl. No.: 13/821,094

(22) PCT Filed: Sep. 8, 2010
(86) PCT No.: PCT/IN2010/000596

§ 371 Date: Mar. 6, 2013

(51) Int. Cl. C07D 277/56

(52) U.S. Cl. CPC C07D 277/56
USPC 548/201

(57) ABSTRACT

Disclosed is a process for the preparation of Ethyl 4-methyl-2-(4-(2-methylpropyloxy)-3-cyanophenyl)-5-thiazolecarboxylate (I) the key intermediate for the preparation of [2-[3-cyano-4-(2-Methyl-propoxy)phenyl]-4-methyl-5-thiazole carboxylic acid (Febuxostat, I(A)) is approved under the trademark Uloric® by the US Food and Drug Administration for the treatment of hyperuricemia and gouty arthritis.

### BACKGROUND OF INVENTION

The preparation of Ethyl 4-methyl-2-(4-(2-methylpropyloxy)-3-cyanophenyl)-5-thiazolecarboxylate is described in EP 0513379 wherein In the following route is described (Scheme-1):

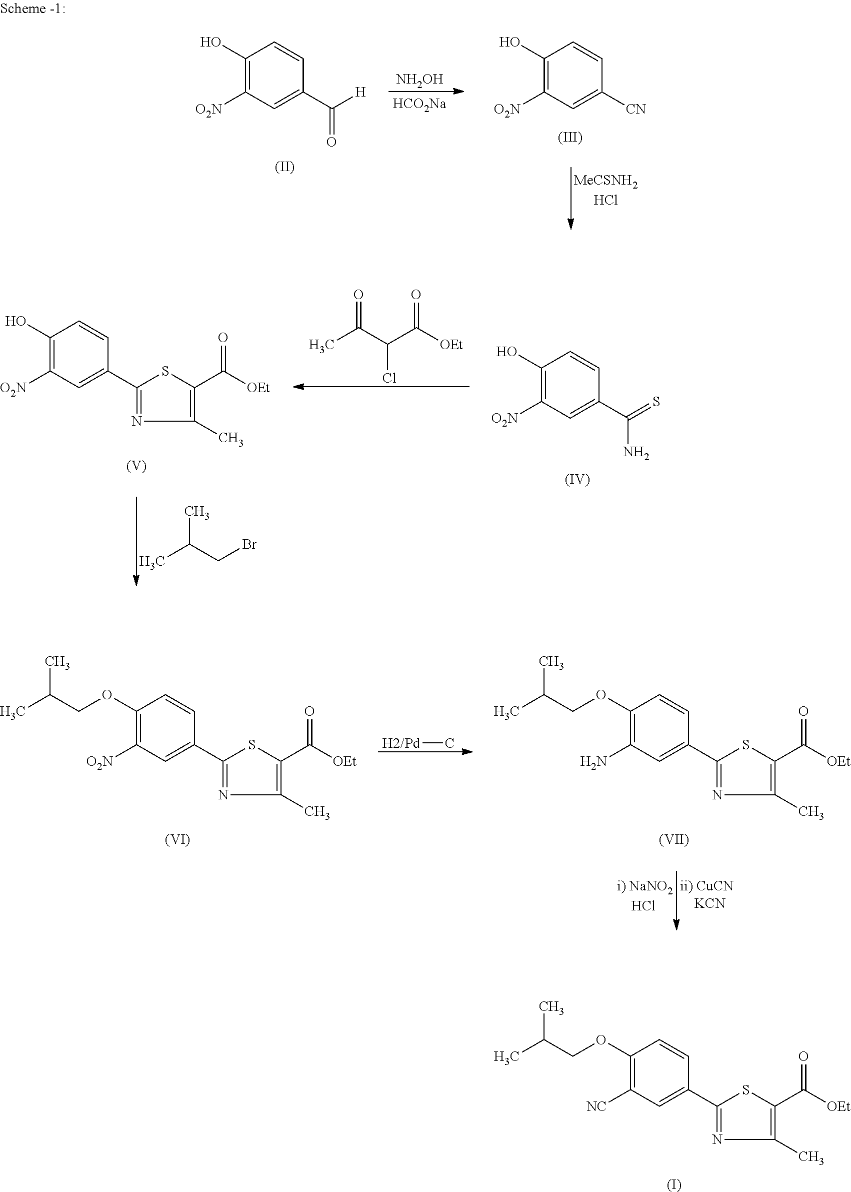

Reaction of 4-hydroxy-3-nitrobenzaldehyde (II) with hydroxylamine and sodium formate in refluxing formic acid gives 4-hydroxy-3-nitrobenzonitrile(III), which is treated with thioacetamide to give corresponding thiobenzamide(IV). The cyclization of (IV) with 2-chloroacetoacid ethyl ester affords 2-(4-hydroxy-3-nitrophenyl)-4-methylthiozole-5-carboxylic acid ethyl ester(V), which is alkylated with isobutyl bromide providing the isobutyl ether(VI). The reduction of the Nitro-group of (VI) with H₂ over Pd/C gives the amino derivative(VII), which is converted into Ethyl 4-methyl-2-(4-(2-methylpropyloxy)-3-cyanophenyl)-5-thiazolecarboxylate(I) by diazotization followed by and treatment with CuCN and KCN

The following are the drawbacks of the process:
The final step is diazotization followed by cyanation involving extremely toxic reagent potassium cyanide.
This cyanation reaction was found to be runaway reaction even on 40 g scale
Further Column chromatography is necessary to purify the product (I)
Cyanation has resulted in low yield (30% of crude product yield)

Another route is disclosed in JP1994/345724 and in Heterocycles 1998, 47: 857-64. This route is illustrated by the following Scheme-2

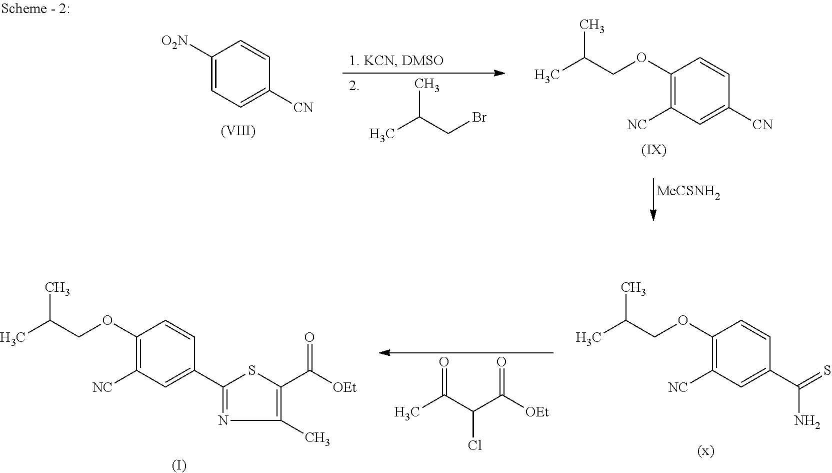

The reaction of 4-nitrobenzonitrile(VIII) with KCN in DMSO in hot DMSO, followed by treatment with isobutyl bromide gives 4-isobutoxybenzene-1,3-dicarbonitrile(IX), which is treated with thioacetamide to yield 3-cyano-4-isobutoxythiobenzamide(X). Cyclization of (X) with 2-chloroacetoacitic acid ethyl ester affords Ethyl4-methyl-2-(4-(2-methylpropyloxy)-3-cyanophenyl)-5-thiazolecarboxylate(I)
The above process involves extremely toxic potassium cyanide
Starting material for this process is expensive
All the three steps require column chromatography for purification

Yet another process is described for the preparation of compound (I) in JP 1998/045733. This route can be illustrated by the following scheme-3

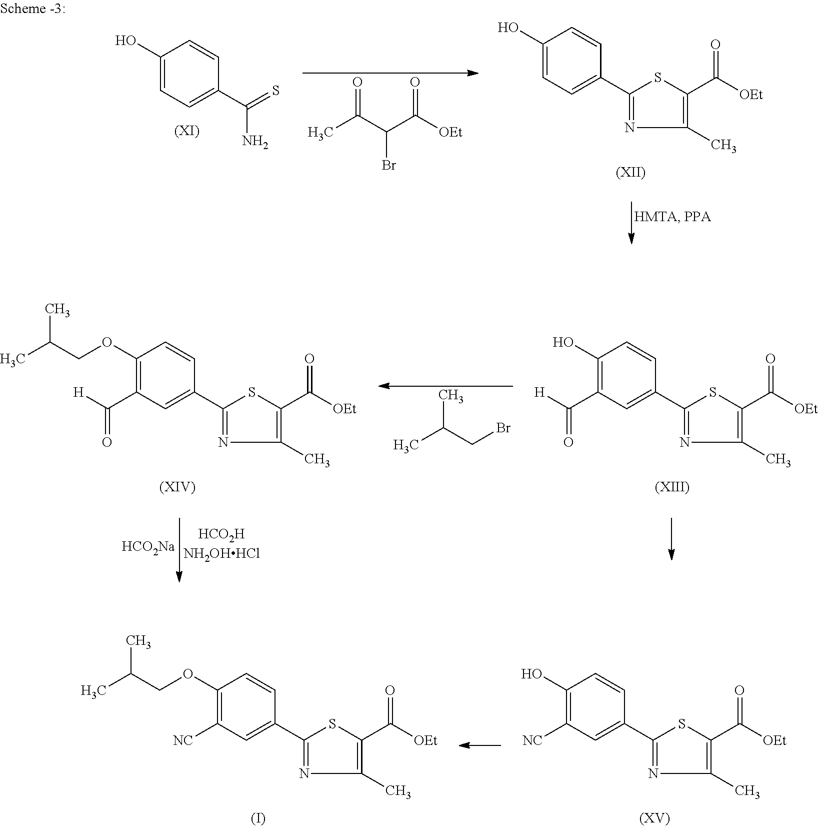

Cyclization of 4-hydroxythiobenzamide(XI) with 3-bromoacetoacetic acid ethyl ester provides 2-(4-hydroxyphenyl)-4-methylthiazole-5-carboxylic acid ethyl ester(XII), which is formylated by reaction with hexamethylenetetramine (HMTA) and polyphosphoric acid to afford 2-(3-formyl-4-hydroxyphenyl)-4-methylthiazole-5-carboxylic acid ethyl ester(XIII). Alkylation of (XIII) with isobutyl bromide gives 2-(3-formyl-4-isobutoxyphenyl)-4-methylthiazole-5-carboxylic acid ethyl ester(XIV), which is treated with formic acid, sodium formate and hydroxylamine hydrochloride to give Ethyl 4-methyl-2-(4-(2-methylpropyloxy)-3-cyanophenyl)-5-thiazolecarboxylate(I). Alternatively 2-(3-formyl-4-hydroxyphenyl)-4-methylthiazole-5-carboxylic acid ethyl ester(XIII) treated with formic acid, sodium formate and hydroxylamine hydrochloride to provide 2-(3-cyano-4-hydroxyphenyl)-4-methylthiazole-5-carboxylic acid(XV), which is treated with isobutyl bromide to give Ethyl 4-methyl-2-(4-(2-methylpropyloxy)-3-cyanophenyl)-5-thiazolecarboxylate(I)
This process also requires column chromatography for the purification of compound of formula (i) from compound of formula (XIV)

It is very important to examine a process of preparing the compound of formula (I) from the point of industrial applicability whether the procedure fulfills the following requirements
1. Commercial availability of starting materials
2. Avoiding toxic/harmful reagents.
3. Environmental compatibility
4. Minimizing byproducts/waste streams
5. Avoiding special equipment requirements
6. Very pure final product and clean impurity profile
7. Overall process economy and commercial viability

All of the processes described above in the prior art do not fulfill one or other of the above conditions.

Further compound of formula (i) is the precursor of Febuxostat. As such, there is a need for compound of formula (I) of high purity which may be conveniently used as a precursor in the preparation of highly pure Febuxostat for therapeutic application.

Therefore we directed our R & D program to develop an improved process for the preparation of compound (I) taking into consideration the above mentioned requirements. The aim being to provide a new environmentally protective, safe, industrially viable process, which is devoid of the insufficiencies of the known procedures and makes possible the synthesis of compound (I) in high yields and purity.

### SUMMARY OF INVENTION

Accordingly we directed our research based on the under mentioned points
Avoiding the usage of potassium cyanide
Avoiding special techniques like column chromatography
Reducing the number of steps
Improving the purity of compound of (I) by hydrochloride salt formation
Improvement of overall yield and process economy

Therefore the main object of the present invention is to provide an improved process for the preparation of highly pure (>99.0%) Febuxostat precursor Ethyl 4-methyl-2-(4-(2-methylpropyloxy)-3-cyanophenyl)-5-thiazolecarboxylate(I) hydrochloride avoiding the drawbacks of the hitherto known processes

Accordingly following scheme-4 illustrates salient aspects of the current invention.

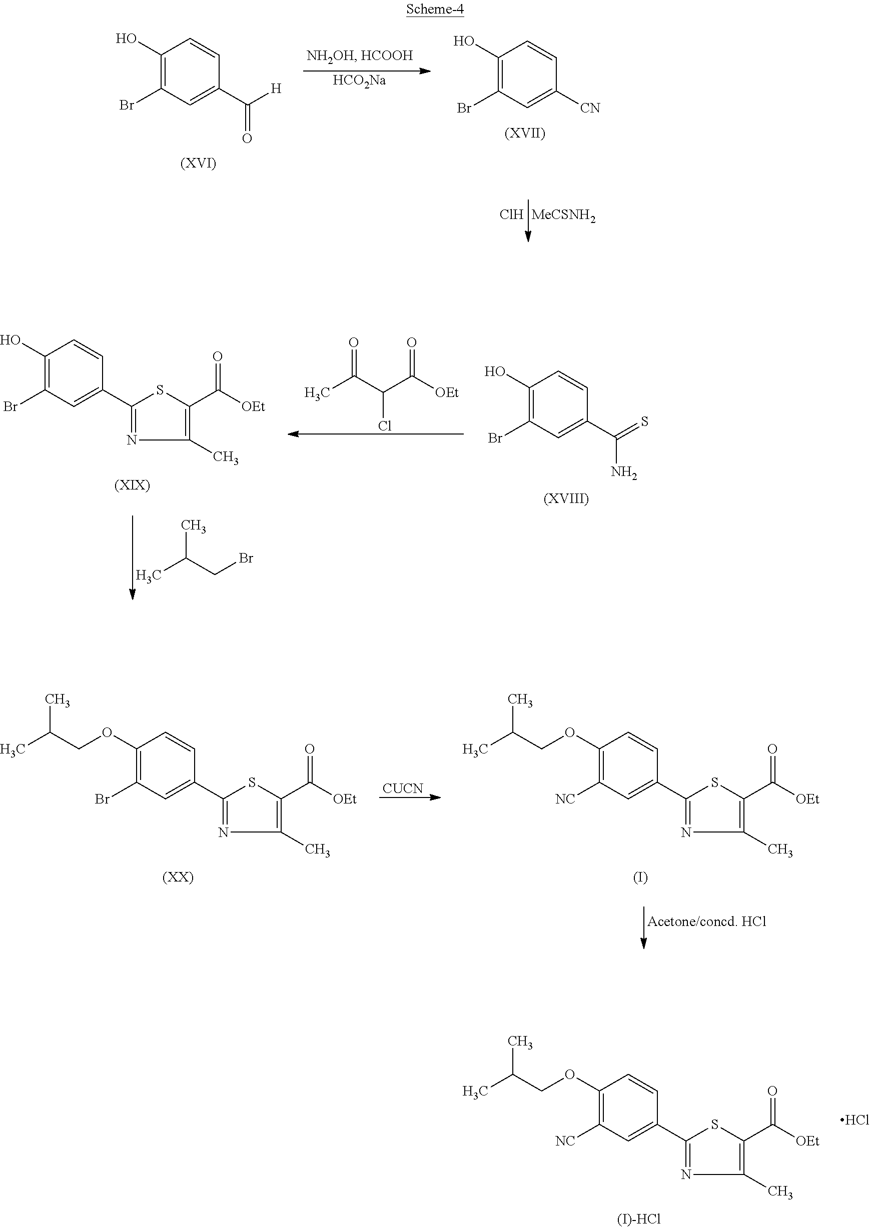

Reaction of 3-bromo-4-hydroxy-benzaldehyde(XVI) with hydroxylamine hydrochloride and sodium formate in refluxing formic acid gives 3-bromo-4-hydroxy-benzonitrile (XVII). Treatment of the compound (XVII) with Thioacetamide gives 3-bromo-4-hydroxy-thiobenzamide(XVIII). Cyclization of compound (XVIII) with 2-chloroacetoacetic acid ethyl ester gives 2-(3-bromo-4-hydroxyphenyl)-4-methylthiazole-5-carboxylic acid ethyl ester(XIX). Alkylation of the compound (XIX) with isobutyl bromide gives 2-(3-bromo-4-isobutoxyphenyl)-4-methylthiazole-5-carboxylic acid ethyl ester(XX). Compound-XX on cyanation with cuprous cyanide gives Ethyl 4-methyl-2-(4-(2-methylpropyloxy)-3-cyanophenyl)-5-thiazolecarboxylate(I). Compound of formula-I is purified further by forming hydrochloride salt in acetone medium

Accordingly, the present invention provides process for the preparation of 3-bromo-4-hydroxy-benzonitrile (XVII) which comprises
Charging 98% formic acid and 3-bromo-4-hydroxy benzaldehyde and stirring for 15 minutes
Charging hydroxylamine hydrochloride and sodium acetate
Heating reaction mass to 105° to 110° C. and maintaining for five hours
Cooling reaction mass to room temperature and adding water and stirring for 2 hours
Filtering followed by drying and taking (XVII) to next stage

Accordingly, the present invention provides process for the preparation of 3-bromo-4-hydroxy-thiobenzamide-(XVIII) which comprises
Charging Isopropyl alcoholic hydrogen chloride to the compound (XVII) and stirring for 15 minutes
Charging thioacetamide and heating to 50-55° C.
Maintaining reaction mass at the same temperature for two hours.
Bringing reaction mass to room temperature
Charging water to the reaction mass and cooling
Filtering, washing with water and drying and taking compound (XVIII) to next stage

Accordingly, the present invention provides an improved method for the preparation of 2-(3-bromo-4-hydroxyphenyl)-4-methylthiazole-5-carboxylic acid ethyl ester(XIX) which comprises
Charging Isopropyl alcohol to the compound of formula (XVIII) and stirring for 5 minutes
Charging Ethyl-2-chloroacto acetate and heating to reflux temperature
Maintaining five hours at reflux temperature
Bringing reaction mass to room temperature
Filtering and drying to yield compound (XIX)

Accordingly, the present invention provides an improved method for the preparation of 2-(3-bromo-4-isobutoxyphenyl)-4-methylthiazole-5-carboxylic acid ethyl ester(XX) which comprises
Charging compound (XIX) and DMF
Charging potassium carbonate and Isobutyl bromide
Heating reaction mass to 80-85° C. and maintaining for six hours
Bringing reaction mass to room temperature and quenching into water
Filtering and washing with water
Suspending wet salt in a mixture of water and Ethyl acetate
Stirring reaction mass for 30 minutes and separating two clear layers.
Extracting aqueous layer with Ethyl acetate and combining organic layers.
Washing Ethyl acetate layer with water and drying over sodium sulphate
Distilling off Ethyl acetate completely and leaching with methanol
Drying to yield compound (XX)

Accordingly, the present invention provides an improved method for the preparation of Ethyl 4-methyl-2-(4-(2-methylpropyloxy)-3-cyanophenyl)-5-thiazolecarboxylate(I) hydrochloride which comprises
Charging compound of formula (XX) and DMF
Charging Cuprous cyanide and cuprous iodide
Heating reaction mass to 130-135° C. temperature and maintaining for 16 hours
Bringing reaction mass to room temperature and quenching into water
Charging ethylene diamine and stirring for 15 minutes

Extracting with Ethyl acetate and washing Ethyl acetate layer with water
Concentrating the solvent and filtering after cooling to 0-5° C.
Drying compound (I) and recrystallization with n-butanol
Drying and suspending dried compound in acetone
Heating to 50° C. to get clear solution followed by cooling the reaction mass to 30-35° C.
Slowly adding Concentrated hydrochloric acid and cooling reaction mass to 0-5° C.
Filtering and drying to yield hydrochloride salt of compound of formula (I)

The solid state properties of Ethyl 4-methyl-2-(4-(2-methylpropyloxy)-3-cyanophenyl)-5-thiazolecarboxylate (I) as hydrochloride salt are illustrated by the following figures:

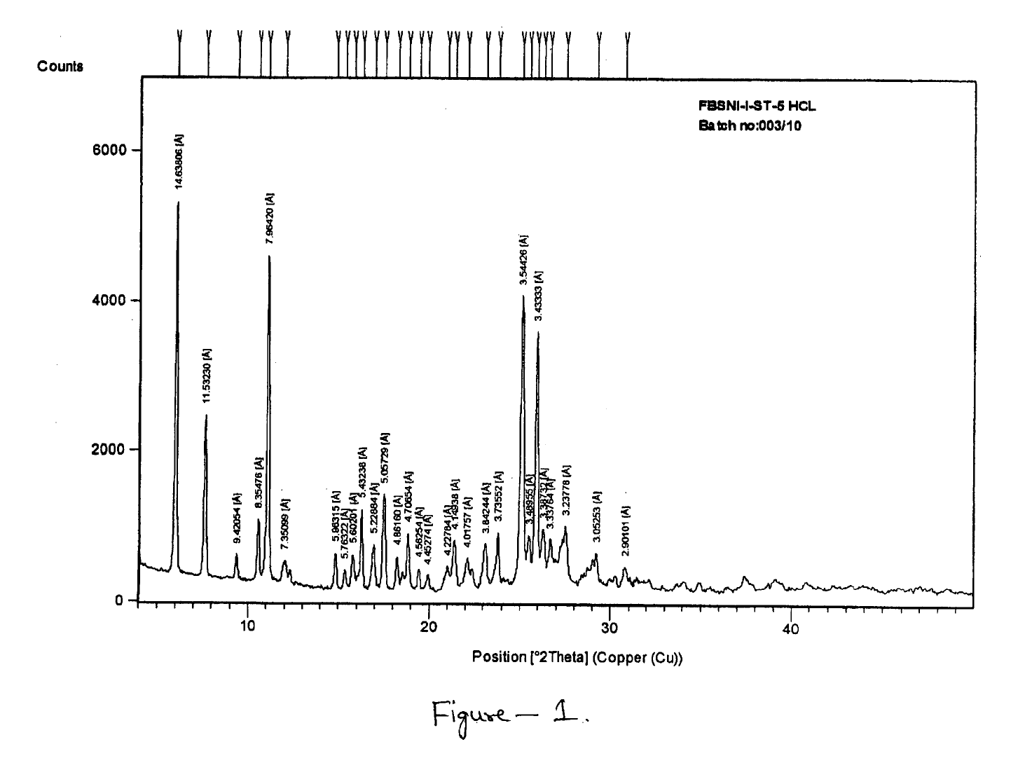

FIG. 1—XRPD spectrum of the hydrochloride salt of compound of the formula-I prepared by the method disclosed in example-1

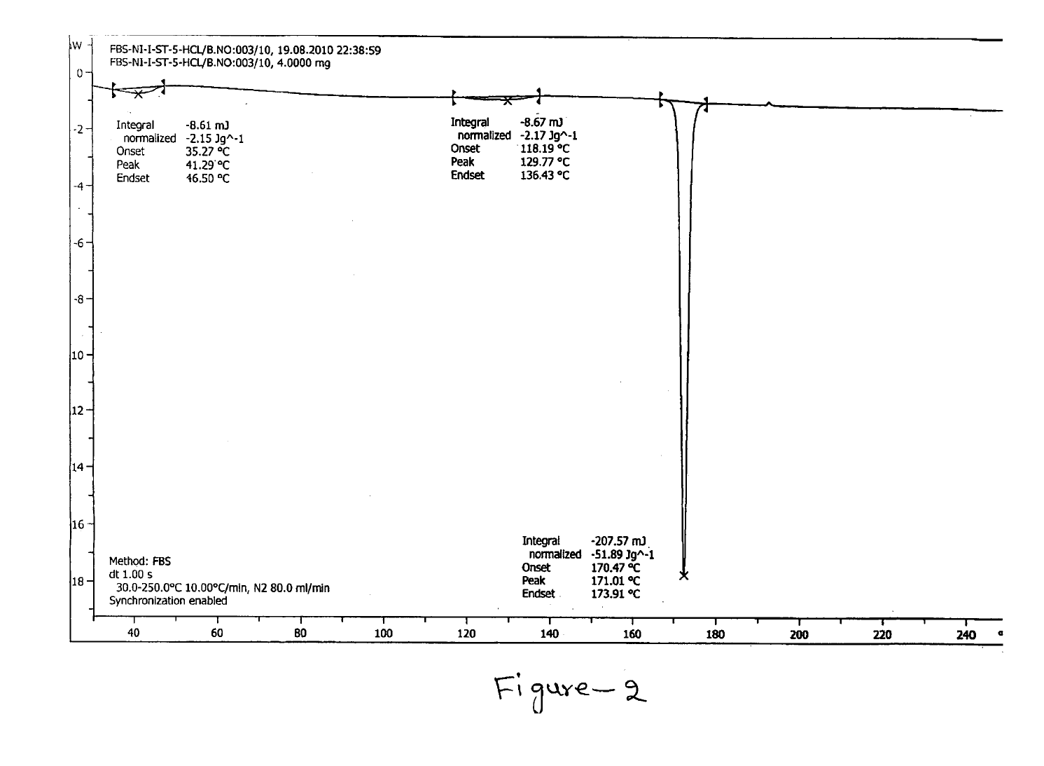

FIG. 2—DSC curve of the hydrochloride salt of compound of the formula-I prepared by the method disclosed in example-1

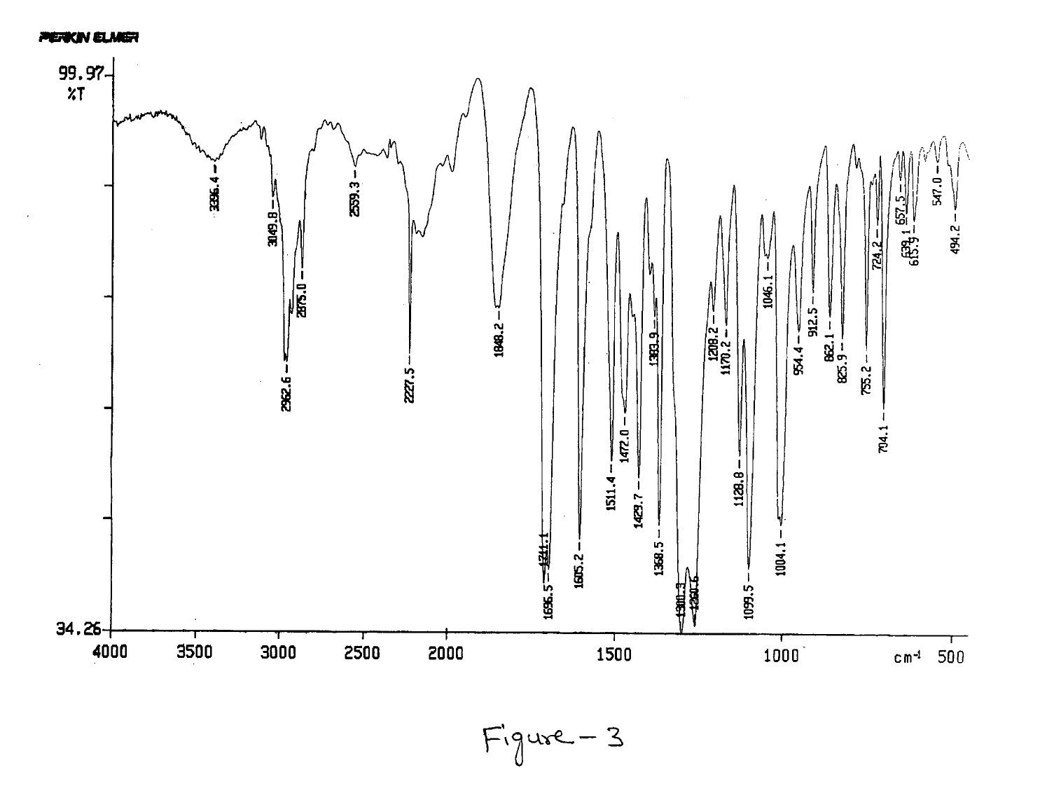

FIG. 3—IR spectrum of the hydrochloride salt of compound of the formula-I prepared by the method disclosed in example-1

The details of the inventions are given in the Examples which are provided for illustration only and therefore the Examples should not be construed to limit the scope of the invention.

### EXAMPLE

### Process for the preparation of Ethyl 4-methyl-2-(4-(2-methylpropyloxy)-3-cyanophenyl)-5-thiazolecarboxylate(I) hydrochloride

### Step-1: Preparation of 3-bromo-4-hydroxy-benzonitrile (XVII)

Into a 3 L round bottomed flask formic acid (98%, 0.7 L) and 3-bromo-4-hydroxy-benzaldehyde (100 g) were charged and stirred for 15 minutes. Sodium formate (59 g) and hydroxylamine hydrochloride (38.4 g) were charged and the reaction mixture was heated to 105-110° C. Reaction mass was maintained at the same temperature for 5 hours and brought to room temperature. Water (2.3 L) was added and the reaction mass was stirred for 2 hours. Reaction mass was filtered and washed with water (500 ml). Dried in tray drier at 60-65° C.

Yield: 69 g (70%)

Purity by HPLC: 97%

Melting range: 150-158° C.

### Step-II: preparation of 3-bromo-4-hydroxy-thiobenzamide(XVIII)

Into a 3 L round bottomed flask a mixture of Isopropanolic hydrogen chloride (124 ml) and compound of formula-XVII (50 g) from the previous step were charged and stirred for 15 minutes. Thioacetamide (33.5 g) was charged and heated to 50-55° C. The reaction mass was maintained at the same temperature for 2 hour and water (330 ml) was added to the and stirred for 2 hours at 5-10° C. The product was filtered and dried at 50-60° C.

Yield: 43 g (75%)

Purity by HPLC: 95%

Melting range: 108-110° C.

### Step-III: Preparation of 2-(3-bromo-4-hydroxyphenyl)-4-methylthiazole-5-carboxylic acid ethyl, ester(XIX)

Into a 1 L round bottomed flask Isopropanol (310 ml) and compound-XIII (40 g) from step-II were charged and stirred for 15 minutes. Ethyl-2-chloro aceto acetate (35.5 g) was charged and the reaction mass was heated to 80-85° C. and maintained at the same temperature for five hours. The reaction mass was brought to room temperature and maintained at the same temperature for 2 hours. The product was filtered and dried at 60-65° C.

Yield: 47 g (90%)

Purity by HPLC: 98.4%

Melting range: 204-210° C.

### Step-IV: preparation of 2-(3-bromo-4-isobutoxyphenyl)-4-methylthiazole-5-carboxylic acid ethyl ester (XX)

Into a 3 L round bottomed flask compound (XIX) from step-III (40 g) and dimethyl formamide (200 ml) were charged. Potassium carbonate (96.9 g) and isobutyl bromide (48.3 g) were added and the reaction mass was heated to 80-85° C. Reaction mass was maintained at the same temperature for five hours and brought to room temperature. Water (2 L) was charged to reaction mass and stirred for one hour. Reaction mass was filtered and washed with water (2×500 ml). The wet compound was dissolved in Ethyl acetate (1000 ml) and ethyl acetate layer was washed with water (400 ml×3). Ethyl acetate layer was dried over sodium sulphate and distilled off completely under vacuum. Methanol (240 ml) was added to the residue and heated to 50-55° C. and maintained at the same temperature for 15 minutes. Reaction mass was brought to room temperature and maintained for one hour. The compound (XX) was filtered and dried at 50-60° C.

Yield: 34 g (73.2%)

Purity by HPLC: 98%

Melting range: 108-109° C.

### Step-V: Preparation of Ethyl 4-methyl-2-(4-(2-methylpropyloxy)-3-cyanophenyl)-5-thiazolecarboxylate(I) hydrochloride

Into a 3 L round bottomed flask compound (XX) from step-IV (34 g) and dimethyl formamide (340 ml) were charged. Cuprous cyanide (13 g) and cuprous iodide (3.4 g) were added to reaction mass and heated to 130-140° C. The reaction mass was maintained at the same temperature for 16 hours, brought to room temperature and quenched into water (6.8 L). It was extracted with Ethyl acetate (3×750 ml) and the organic layer was washed with water (1.51×2). The organic layer was dried over sodium sulphate and the solvent was distilled off completely under vacuum. Ethyl acetate (155 ml) was added to the residue and cooled to 0-5° C. and maintained at the same temperature for 30 minutes. The reaction mass was filtered and recrystallized with n-butanol (700 ml). The compound-I was dried at 60-70° C. Yield: 22.5 g (98.5% by HPLC).

### Hydrochloride Salt of Compound-I:

Compound of formula-I was suspended in acetone (660 ml) and heated to 50° C. and maintained at the same temperature for 15 minutes. Clear solution was brought to 30-35° C. and Concentrated hydrochloric acid (20 ml) was added slowly during 30 minutes. The reaction mass was cooled to 0-5° C. and filtered. The product was dried at 60-70° C.

Yield: 20 g (61.5%)

Purity by HPLC: 99.5%

Melting range: 170-173° C.

This hydrochloride salt can be directly taken for next hydrolysis step to get pharmaceutical grade Febuxostat.

### ADVANTAGES OF THE INVENTION

1) Ethyl4-methyl-2-(4-(2-methylpropyloxy)-3-cyanophenyl)-5-thiazolecarboxylate of formula (I) is produced in more than 99.0% chemical purity.
2) Ethyl4-methyl-2-(4-(2-methylpropyloxy)-3-cyanophenyl)-5-thiazolecarboxylate of formula (I) prepared by this method is suitable for synthesis of pharmaceutical grade Febuxostat.

We claim:

1. Improved process to prepare for the preparation of Ethyl 4-methyl-2-(4-(2-methylpropyloxy)-3-cyanophenyl)-5-thiazolecarboxylate of the formula-(I) as hydrochloride salt 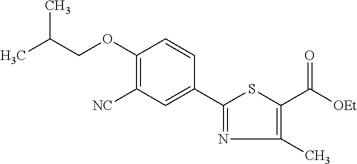 [SMILES: CCOC(=O)c1sc(-c2ccc(OCC(C)C)c(C#N)c2)nc1C]
Comprising the following steps:
a. Reaction of 3-bromo-4-hydroxy-benzaldehyde(XVI) with hydroxylamine and sodium formate in refluxing formic acid affords 3-bromo-4-hydroxy-benzonitrile (XVII). 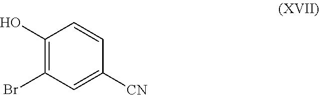
b. Treatment of compound (XVII) with thioacetamide giving rise to 3-bromo-4-hydroxy-thiobenzamide (XVIII). 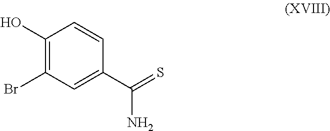 [SMILES: NC(=S)c1ccc(O)c(Br)c1]
c. Cyclization of compound (XVIII) with 2-chloroacetoacetic acid ethyl ester affording 2-(3-bromo-4-hydroxyphenyl)-4-methylthiazole-5-carboxylic acid ethyl ester(XIX). 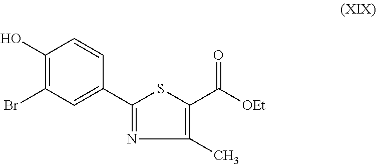 [SMILES: CCOC(=O)c1sc(-c2ccc(O)c(Br)c2)nc1C]
d. Alkylation of compound (XIX) with isobutyl bromide gives 2-(3-bromo-4-isobutoxyphenyl)-4-methylthiazole-5-carboxylic acid ethyl ester(XX). 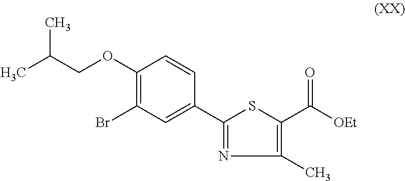 [SMILES: CCOC(=O)c1sc(-c2ccc(OCC(C)C)c(Br)c2)nc1C]
e. Compound XX on cyanation with cuprous cyanide affords Ethyl 4-methyl-2-(4-(2-methylpropyloxy)-3-cyanophenyl)-5-thiazolecarboxylate(I).
f. Purification of compound I by forming hydrochloride salt I—HCl in acetone medium

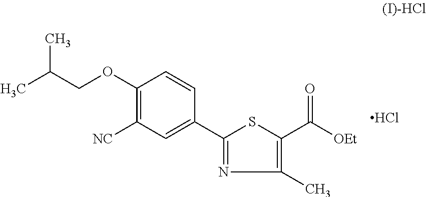 [SMILES: CCOC(=O)c1sc(-c2ccc(OCC(C)C)c(C#N)c2)nc1C ; Cl]

2. Process as claimed in claim 1 wherein the step(a) comprises:
i. Charging 98% formic acid and 3-bromo-4-hydroxy benzaldehyde and stirring for 15 minutes
ii. Charging hydroxylamine hydrochloride and sodium acetate
iii. Heating reaction mass to 105° to 110° C. and maintaining for five hours
iv. Cooling reaction mass to room temperature and adding water and stirring for 2 hours
v. Filtering followed by drying and taking (XVII) to next stage

3. Process as claimed in claim 1 wherein the step(b) comprises:
i. Charging Isopropyl alcoholic hydrogen chloride to the compound (XVII) and stirring for 15 minutes
ii. Charging thioacetamide and heating to 50-55° C.
iii. Maintaining reaction mass at the same temperature for two hours
iv. Bringing reaction mass to room temperature
v. Charging water to the reaction mass and cooling
vi. Filtering, washing with water and drying and taking compound (XVIII) to next stage

4. Process as claimed in claim 1 wherein the step(c) comprises:
I. Charging Isopropyl alcohol to the compound of formula (XVIII) and stirring for 5 minutes
II. Charging Ethyl-2-chloroacto acetate and heating to reflux temperature
III. Maintaining five hours at reflux temperature
IV. Bringing reaction mass to room temperature
V. Filtering and drying to yield compound (XIX)

5. Process as claimed in claim 1 wherein the step(d) comprises:
I. Charging compound (XIX) and DMF
II. Charging potassium carbonate and Isobutyl bromide
III. Heating reaction mass to 80-85° C. and maintaining for six hours
IV. Bringing reaction mass to room temperature and quenching into water
V. Filtering and washing with water
VI. Suspending wet salt in a mixture of water and Ethyl acetate
VII. Stirring reaction mass for 30 minutes and separating two clear layers.
VIII. Extracting aqueous layer with Ethyl acetate and combining organic layers.
IX. Washing Ethyl acetate layer with water and drying over sodium sulphate
X. Distilling off Ethyl acetate completely and leaching with methanol
XI. Drying to yield compound (XX)

6. Process as claimed in claim 1 wherein the step(e) comprises:
I. Charging compound of formula (XX) and DMF
II. Charging Cuprous cyanide and cuprous iodide
III. Heating reaction mass to 130-135° C. temperature and maintaining for 16 hours
IV. Bringing reaction mass to room temperature and quenching into water
V. Charging ethylene diamine and stirring for 15 minutes
VI. Extracting with Ethyl acetate and washing Ethyl acetate layer with water
VII. Concentrating the solvent and filtering after cooling to 0-5° C.
VIII. Drying compound (I) and recrystallization with n-butanol
IX. Drying and suspending dried compound in acetone
X. Heating to 50° C. to get clear solution followed by cooling the reaction mass to 30-35° C.
XI. Slowly adding Concentrated hydrochloric acid and cooling reaction mass to 0-5° C.
XII. Filtering and drying to yield hydrochloride salt of compound of formula(I)

7. A method of preparing ethyl 4-methyl-2-(4-(2-methylpropyloxy)-3-cyanophenyl)-5-thiazolecarboxylate of formula (I) as hydrochloride salt essentially as in example-1

8. A method of preparing ethyl 4-methyl-2-(4-(2-methylpropyloxy)-3-cyanophenyl)-5-thiazolecarboxylate of formula (I) as hydrochloride as in claims 1-7 and having purity of more than 99.0%

9. A method of preparing Ethyl 4-methyl-2-(4-(2-methylpropyloxy)-3-cyanophenyl)-5-thiazolecarboxylate of formula (I) as hydrochloride salt as in claims 1-8 and having solid state characteristics as in FIGS. 1-3.
